(19) United States
(12) Patent Application Publication
(10) Pub. No.: US 2014/0162338 A1
(43) Pub. Date: Jun. 12, 2014

(54) DEVICE AND METHOD FOR PRODUCING A COLD, HOMOGENEOUS PLASMA UNDER ATMOSPHERIC PRESSURE CONDITIONS

(71) Applicants: Jan Schaefer, Greifswald (DE); Stefan Horn, Greifswald (DE); Ronny Brandenburg, Gross-Kiesow (DE); Ruediger Foest, Neuenkirchen (DE); Manfred Stieber, Greifswald (DE); Klaus-Dieter Weltmann, Binz (DE)

(72) Inventors: Jan Schaefer, Greifswald (DE); Stefan Horn, Greifswald (DE); Ronny Brandenburg, Gross-Kiesow (DE); Ruediger Foest, Neuenkirchen (DE); Manfred Stieber, Greifswald (DE); Klaus-Dieter Weltmann, Binz (DE)

(73) Assignee: Leibniz-Institut fuer Plasmaforschung und Technologie e.V., Greifswald (DE)

(21) Appl. No.: 14/122,896

(22) PCT Filed: May 29, 2012
(86) PCT No.: PCT/EP2012/059939

§ 371 Date: Feb. 19, 2014

(30) Foreign Application Priority Data
May 31, 2011 (DE) 10 2011 076 806.8

(51) Int. Cl. H05H 1/24; B01J 19/08; A61L 2/03

(52) U.S. Cl. CPC H05H 1/2406; A61L 2/03; B01J 19/088
USPC 435/173.1; 315/111.21; 422/22; 216/67; 204/164

(57) ABSTRACT

The invention relates to a special plasma source designated as a plasma intractor (PI) for producing a cold, homogeneous plasma under atmospheric pressure conditions, which plasma source can be used advantageously to excite and control reactive processes in flowing media. The device according to the invention is characterized in that the device comprises at least 6 elongated electrodes (2) and a molded body (1) made of insulating material, the molded body (1) being provided with an elongated cylindrical cavity (7) and with additional holes, which are guided parallel to the cavity (7) and arranged symmetrical to the axis of the cavity and equidistant to one another, and the electrodes (2) are embedded in holes of the molded body (1) and are connected to an AC high-voltage supply in such a way that the polarities of respective adjacent electrodes are opposite in each phase of the voltage period.

The invention relates to a special plasma source known as a plasma intractor (PI) for generating a cold, homogeneous plasma under atmospheric pressure conditions according to the preamble of claim 1, which source can be used advantageously for excitation and control of reactive processes in flowing media.

By the term “cold” plasma there is understood a nonthermal low-temperature plasma, which is characterized by the following features:
(i) the neutral temperature of the gas of the plasma is comparable to or only slightly higher than the ambient temperature (i.e. laboratory temperature, or temperature of the device or temperature of the working gas before ignition of the plasma) and
(ii) the device is able to generate the plasma continuously for several hours without any active cooling system

By the term “homogeneous” plasma there is understood here a discharge structure, generated by the device and averaged over the voltage period, that has surprisingly small fluctuations of ? radiation intensity in the visible spectral region, especially in the axial direction relative to the electrode assembly. This property represents a major advantage compared with the filamented structure of common dielectrically hindered discharges under atmospheric pressure.

### PRIOR ART

Numerous plasma sources and plasma technological processes for cleaning or activation of surfaces under atmospheric pressure conditions are described in the technical or patent literature (review article: K. H. Becker et al., J. Phys. D: Appl. Phys. 39 (2006), R55; M. Laroussi et al., Plasma Process. Polym. 4 (2007), 777 as well as F. Iza et al., Plasma Process. Polym. 5 (2008), 322). When a medium suitable for forming coatings (precursor) is injected in addition to the process gas into the plasma zone, plasma sources of this type may also be used for plasma-assisted coating of surfaces, with the goal of permanently improving the surface properties of a material in terms of chemical resistance, wettability, adhesion, scratch resistance, gas permeability, tribological, optical and dielectric parameters, etc. (examples: J. Jan\check{c}a et al., Surf. Coat. Technol. 547 (1999), 116; J. Schäfer et al., Plasma Process. Polym. 6 (2009), S519, J. Schäfer et al., Eur. J. Phys. D 54 (2009), 211; Review article: C. Tendero et al., Spectrochimica Acta Part B 61 (2006) 2; L. Bárdos et al., Thin Solid Films 518 (2010), 6705; Patents: U.S. Pat. No. 006,525,481 B1, EP 2209354 A2, WO 2008/074604 A1, WO 2006/092614 A2, WO 2009/073292A1, WO 2009/037331 A1, WO 2009/031886 A2).

The technical solutions of Patents: DE 12059831B4, DE 102007030915A1, DE 10116502A1, US 2009/01888626A1, EP 1375851A1, DE 19534950A1, WO 98/35379) have the following disadvantages:
(a) Position of the electrodes relative to the geometry of the supply of medium:

In contrast to the inventive arrangement, the electrodes are mostly either of axial and central construction (points, needles, etc.) or of radial construction (e.g. annular electrodes). In the first case, the flow of medium around the electrodes leads to disadvantageous flow dynamics (turbulence). In the second case, axial gradients of electric field strength develop, thus preventing an elongated axial source geometry.
(b) Insulation of the electrodes:

Conventional plasma jets often have metallic electrode surfaces, which are in direct contact with the plasma. For a surface treatment, this configuration has proved to be disadvantageous, since electrode erosion allows metal to be transported to the surface to be treated, thus leading to undesired contamination. In addition, increased erosion leads to increased wear and shortened maintenance intervals for plasma sources. Embedding the electrodes in an electrically insulating, robust material proves to be advantageous with respect to the said erosion. However, in the conventional flat geometry (planar or coplanar discharges), at least half of the energy of the electric field is absorbed in the dielectric, and the heat load due to the plasma source must be actively compensated there.
(c) Number of electrodes:

Technical solutions using electrode arrays are known. These are used mostly to increase the area or volume of the entire discharge arrangement. The possibility of finding an optimum for the number of electrodes in the spatial structuring of the electric field, especially in the azimuthal direction in the gas space, is disregarded.

The physical effect of these aspects is involved synergetically in the disadvantage spatial inhomogeneity resulting from filamentation of the plasma and in the development of local physical instabilities of the plasma. This plays a decisive role for process control, when a medium is introduced into the plasma. In conventional plasma sources that generate the plasma under atmospheric pressure conditions in the form of a jet, the medium (for example, a precursor) is admixed directly with the process gas (hereinafter referred to as source gas) or flows through a filamented, primary plasma. In these cases, the reactive process in the medium is space- and time-dependent and accordingly heterogeneous. As a consequence of the heterogeneity, low efficiency of the process is observed in the entire flow cross section of the medium. In practice, the deficit is compensated only partially by stochastic homogenization of the primary plasma or by removal of reaction products from the medium in the immediate vicinity of the primary plasma. Either increased powers or increased flow velocities are suggested as solutions.

In the proposed solution based on increasing the power, the use of the source causes increased thermal load, or the source can be used only in a very restricted zone. From the example of coating of thermally labile surfaces, it can be shown that prolonged action of conventional plasma sources degrades the surface and short-term action causes a strong dependence of the coating quality on the distance between source and surface. At the same time, a homogenizing power increase represents a further restriction for the low-energy processes in the plasma, such as, for example, selective modification of larger precursor molecules, which may be largely destroyed in a high-energy discharge.

In the approach to a homogenization solution based on increasing the flow, the plasma in such sources is made to exit the nozzle in the form of a jet. This jet discharge causes highly turbulent mixing of the plasma with the surrounding atmosphere and thereby is made sensitive to the ambient conditions. Furthermore, processes with longer time scale in the plasma (e.g. processes of collisions with metastable particles) are also restricted. These effects limit the achievable quality of coating and surface modification, and therefore their areas of application are also limited. It is possible that processes requiring conditions that reduce the homogeneous gradient under atmospheric pressure cannot be implemented.

### OBJECT OF THE INVENTION

The object underlying the invention is to eliminate the said major disadvantages of the solutions described in the prior art. It relates in particular to novel compensation of the filamentation of the plasma under atmospheric pressure, since this results in strong gradients of the plasma parameters and gas temperature and also in turbulence of the gas flow. The approach to this solution is intended to lead to achievement of a symmetric homogenized discharge structure, which is highly efficient as regards interaction with the medium to be treated in the cross section of the reactive plasma channel and which leads to high reproducibility of the processes relevant to application even under laminar flow rates and with small coupled energy flows.

### Achievement of the Object

The object was achieved by construction of a special plasma source known as a plasma intractor (PI) for generating a cold, homogeneous plasma under atmospheric pressure conditions according to the features of the protective claims.

### DESCRIPTION OF THE INVENTION

The inventive device for excitation and control of reactive processes in flowing media is a plasma source known as a plasma intractor (PI).

The mode of operation of the plasma source PI is based in general on the principle of coplanar dielectrically hindered discharge (DHD) in a dielectric, preferably ceramic nozzle molded body (1). The DHD is produced in a gas (source gas 6) flowing through the nozzle in cavity (7) under atmospheric conditions. If a suitable medium (5) is mixed in dosed manner into the plasma generated by the DHD (referred to hereinafter as primary plasma 10) through a capillary (4) or tube (the diameter of the arrangement can be scaled from mm to cm in order of magnitude) disposed preferably concentrically in the nozzle, reactive processes are excited in the medium (5). In the case of a gaseous medium (5), this excitation leads to ignition of a secondary discharge (11a) in the medium (5), which flows as effluent (11b) from the capillary (4). The process of coupling of the primary homogeneous plasma into the flowing medium (5) and the resulting generation of the secondary plasma (10) in the medium is the core mechanism of plasma source PI and is known as “plasma intraction”.

High stability, homogeneity and efficiency of plasma intraction is achieved by a new configuration of the electric field in the cavity. In contrast to conventional plasma sources, electrodes (2) are disposed in equidistant, eccentric and elongated relationship in molded body (1), so that the highest field strength of the exciting field is preferably localized in cavity (7). This property leads to an enormous increase in efficiency of the PI. Furthermore, the symmetric arrangement of electrodes (2) ensures optimum distribution of the field strength in the cross section of cavity (7), wherein an elongated mounting of electrodes (2) parallel with the axis of cavity (7) produces gradient-free conditions over greater lengths in axial direction. The number of electrodes is even, while the optimum number of the electrodes can be adapted to the diameter of cavity (7). In nozzles with inside diameter greater than or equal to 4 mm, the minimum number of electrodes for the desired effect is equal to six.

The reactivity of secondary plasma (11) in effluent (11b) is significantly influenced by the energy input released from primary plasma (10) and by the composition of the flowing medium. The coupling can be controlled by the position of the capillary in the nozzle and by the flow velocity of medium (5). The reactive plasma gas mixture can be used for diverse intended applications (e.g. for deposition of thin coatings on the order of nm to pm, surface functionalization of plastics, precision cleaning or even etching).

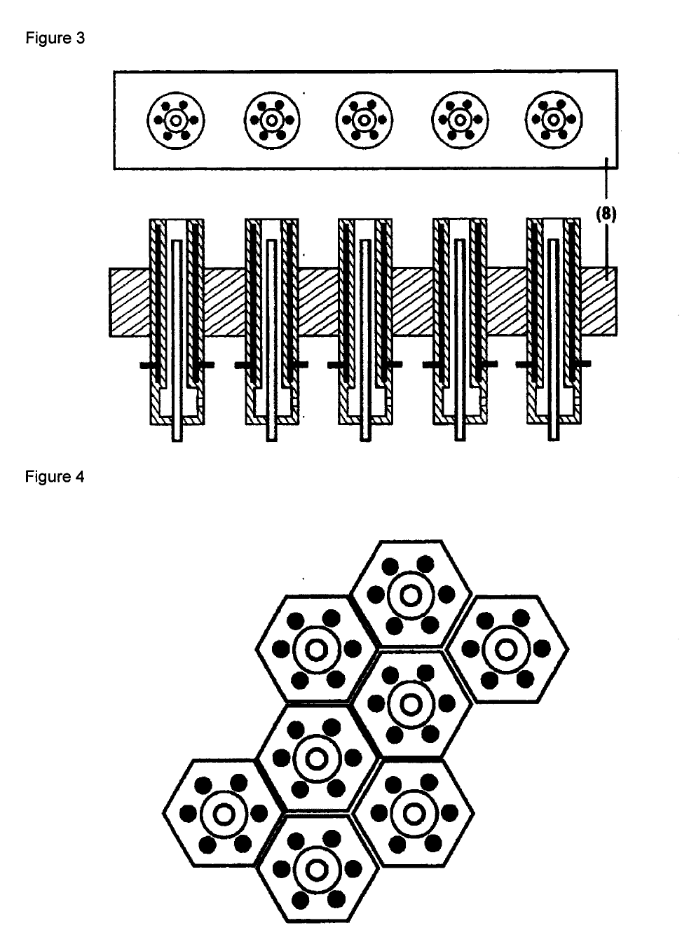

For excitation of primary plasma (10), the electrodes are biased in pairs in the molded body (see FIG. 2). One half of the electrodes is grounded, while an alternating voltage is applied to the other. The polarity of adjacent electrodes is opposite in each phase of the voltage. The possible configurations of the electrodes and different shapes and arrangements of the nozzles will be explained on the basis of corresponding drawings of exemplary embodiments (FIGS. 3-5). In the azimuthal section of the nozzle, the configuration resembles a coplanar plasma source. However, the plane coplanar plasma sources require higher electrode potentials during operation, whereby a larger fraction of the energy is absorbed in the dielectric, and typically the source must be actively cooled. In contrast, the symmetric eccentric “rolled up” electrode configuration of the PI favors coupling of the electric field in the interior zone of the nozzle with substantially higher efficiency. Consequently the generation of the primary plasma is no longer accompanied by thermal load. It takes place at lower voltage than in the case of a comparable coplanar plasma source (US Patent Appl. No. 2004/0194223 A1 or U.S. Pat. No. 4,652,318 A), and the source itself does not need any active cooling. Furthermore, the electrodes of the PI remain free of contact, as is typical of coplanar plasma sources. The avoidance of contact of metallic surfaces with the plasma (freedom of the electrodes) reduces metallic contamination of the plasma and of medium (5) to be processed by material eroded from the electrode surface, and also reduces the complexity associated with maintenance of the PI.

Properties of coplanar DHD and plasma jets are advantageously combined in the PI, in order to influence the homogeneity of the plasma in the length and the stability of the plasma in the cross section of the nozzle to a particularly positive extent. By virtue of the combination of the symmetric spatial structure of the plasma on the inside wall of cavity (7) and of the laminar axial flow of gas (6), a synergy effect is created, contributing to stabilization and homogenization of the plasma. In this case, the streamlines of the gas are oriented perpendicular to the electric field lines. Thereby the concentrations of long-lived or charged plasma species are uniformly distributed axially. Surprisingly, the discharge structure is visually homogeneous axially even in those source gases that exhibit characteristic filamentation under comparable conditions in other conventional plasma sources.

Because of the homogenizing synergy effect in the PI, the effective boundaries of the discharge structure are expanded and radial transport of the charge carriers in the nozzle can take place from the nozzle wall (10) to the axis of the nozzle. If a medium (5) is introduced into the middle of nozzle (1) through a central capillary (4), a secondary plasma is generated in the medium in the capillary under favorable conditions (flow and chemical composition of the medium). The secondary plasma (11) in the gaseous medium is coupled (“intracted”) to the flow of medium (5), and the reactivity of the medium can be surprisingly carried further away, out of the nozzle. The position in the capillary of the nozzle and the flow parameters of the medium are then decisive for the extent of plasma infraction.

The effect of plasma intraction has several advantages relevant for application. The separately adjustable flow velocity of medium (5) permits efficient regulation of the transport of reaction products without any change in the properties of primary plasma (10). This permits a stable process with slight influence of the distance from the nozzle to the target (the position of removal of reaction products). A homogenized and cylindrically symmetric interaction between primary plasma (10) and medium (5) leads to high efficiency of the process. From the example of layered deposition of SiOₓ coatings by means of a PI prototype, an unexpectedly high yield was observed thereby, representing savings in time and cost in practice.

A further substantial advantage of the device consists in the low temperature of the secondary plasma. Because of the fact that the secondary plasma is generated not directly in the medium by the primary electric field strength, but instead by the high density of already generated ionizable species of the primary plasma, the mean kinetic temperature of the secondary plasma is largely determined by the neutral temperature of the medium. This also permits processes with thermally labile or biological materials.

### EXEMPLARY EMBODIMENTS

The invention will be explained in more detail hereinafter on the basis of some examples, without limiting the invention to these examples.

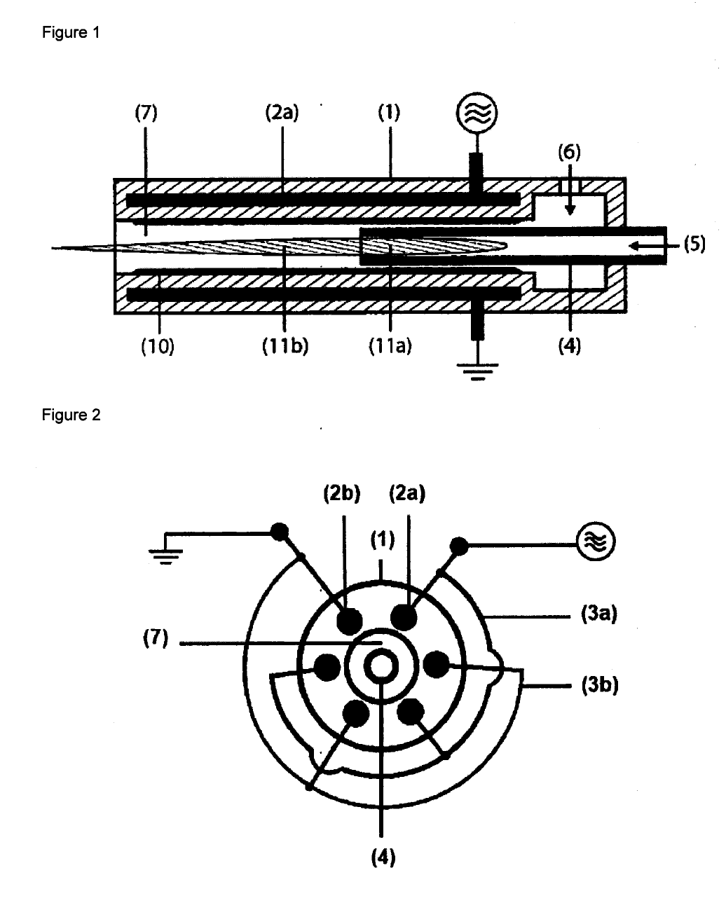

FIGS. 1, 2, 3, 4 and 5 show examples of the inventive device.

The reference numerals below are used hereinafter in the drawings:
(1) Molded body (e.g. ceramic nozzle)
(2) Electrodes (2a: high-voltage electrode, 2b: grounded electrode)
(3) Connecting line (3a: of the high-voltage electrodes; 3b: of the grounded electrodes)
(4) Capillary (e.g. quartz capillary)
(5) Supply of medium
(6) Gas flow (process gas or “source gas”)
(7) Cavity
(8) Base plate
(9) Channel for additional supply of medium
(10) Primary plasma
(11) Secondary plasma, 11a—generation, 11b—effluent

### EXAMPLE 1

### Preferred Embodiment of an Individual PI

FIG. 1 shows the schematic diagram of the nozzle arrangement of an individual PI in longitudinal section and FIG. 2 shows the same arrangement in front view.

The core piece of the PI is a molded body (1) with six recesses for electrodes (2) and an axial cylindrical cavity (7). This cavity (7) serves as a gas space for generation of primary plasma (10). The electrodes (2a and 2b) are embedded in the further six recesses, which are arranged equidistantly from one another around the cavity. Coaxially inserted ceramic capillary (4) is used to supply a medium (5), for example an aerosol comprising a carrier gas and a precursor in the case of an application for coatings. Process gas (6) flows through a corresponding opening at the rear end of the nozzle into cavity (7). Primary plasma (10) is localized on the wall of the cavity, while secondary plasma (11) is produced in the axial zone of capillary (4) and has an effect that extends further to the outlet from the nozzle.

FIG. 2 demonstrates the symmetric arrangement of high-voltage electrodes (2a) and grounded electrodes (2b), which are externally contacted via corresponding connecting lines (3a and 3b) for the high voltage electrodes and for the grounded electrodes. This preferred embodiment of the invention can be used effectively as the PI under the following conditions: working gas: argon, operating frequency of a few kHz to MHz, operating voltage at 10 kHz of 5 to 15 kVpp, corresponding power already of 1 to 10 W per nozzle (nozzle length 2 cm).

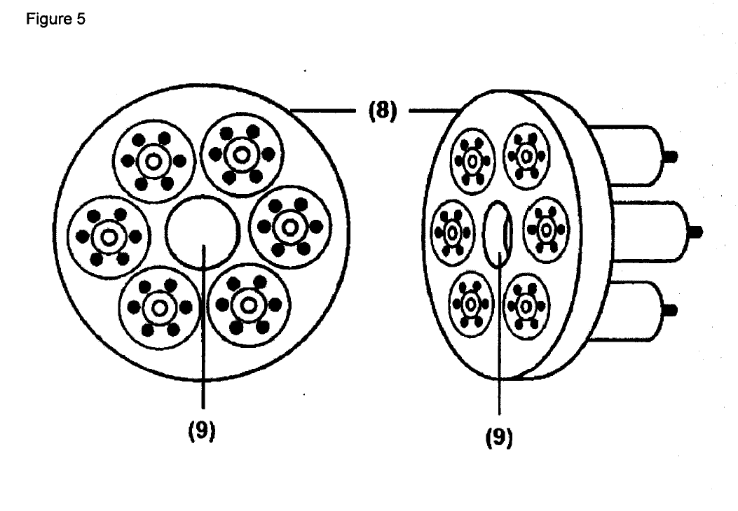

The following figures show possible combinations of several individual PIs in an array (FIG. 3), in a matrix (FIG. 4) and in a concentric configuration (FIG. 5). In these cases, the nozzles are respectively mounted on a correspondingly shaped base plate (8). In the case of the concentric arrangement (FIG. 5), the central channel (9) can be used for supply of further medium.

Example 2 shows a schematic diagram of a linear arrangement of five PIs.

Example 3 shows a schematic diagram of a matrix-type arrangement of several PIs.

Example 4 shows the schematic diagram of a concentric arrangement of six PIs.

1. A device for generating a cold, homogeneous plasma under atmospheric pressure conditions, said device comprising:
at least six longitudinally constructed electrodes, and
a molded body of insulating material,
wherein the molded body is provided with an elongated cylindrical cavity and with further recesses oriented parallel to the cavity and disposed symmetrically relative to the axis of the cavity and equidistant from one another, and
wherein the electrodes are embedded in recesses of the molded body, and
wherein the electrodes are connected in such a way to an AC high voltage supply that the polarities of respective adjacent electrodes are opposite in every phase of the voltage period.

2. The device according to claim 1, wherein a capillary, which is adjustable in axial position and which comprises insulating material, is disposed in the cavity of the molded body.

3. The device according to claim 1, wherein a workpiece to be treated can be introduced and processed in the cavity of the molded body.

4. The device according to claim 3, wherein the workpiece is at least one member selected from the group consisting of a rod, a wire, a braid, a cable, a tube and a fiber.

5. The device according to claim 1, wherein at least one member selected from the group consisting of gases, vapors and liquids is introduced via the capillary into the plasma.

6. A method for generating a homogeneous plasma comprising:
generating a primary plasma in the cavity according to the principle of DHD; and
generating a secondary plasma separately using a dielectric capillary in the cavity, through which the gaseous medium is flowing;
wherein said homogeneous plasma is generated under atmospheric pressure conditions according to the principle of DHD.

7. The method according to claim 6, wherein a reactivity of the secondary plasma is controlled by
the energy from the primary plasma or
by the composition of the flowing medium in the capillary or
the position of the capillary in the cavity.

8. The method according to claim 6, wherein the adaptation of the chemical and physical properties of the secondary plasma is used for the treatment of biological material.

9. A method for external surface modification, comprising:
generating a cold, homogeneous plasma under atmospheric pressure conditions using the device according to claim 1; and
modifying said external surface.

10. An array or matrix, comprising:
at least two devices according to claim 1.

11. The method according to claim 8, wherein the biological material is at least one member selected from the group consisting of tissue, wounds, living cell tissue, biological cells and biological systems.

12. A method for generating an active chemical compound or reactive particle for modification of a surface, comprising:
generating a cold, homogeneous plasma under atmospheric pressure conditions using the device according to claim 1; and
generating said active chemical compound or said reactive particle.

13. A method for internal reactive modification of a heterogeneous substance in a flowing medium, comprising:
generating a cold, homogeneous plasma under atmospheric pressure conditions using the device according to claim 1; and
internally modifying said heterogeneous substance in said flowing medium.

14. A method for plasma-assisted synthesis of a complex product from a flowing medium, comprising:
generating a cold, homogeneous plasma under atmospheric pressure conditions using the device according to claim 1; and
synthesizing at least one complex product selected from the group consisting of clusters, nanoparticles, and chemical compounds, from at least one flowing medium selected from the group consisting of reactive gas mixtures, smoke and aerosols.

15. A method for conversion of a complex medium, comprising:
generating a cold, homogeneous plasma under atmospheric pressure conditions using the device according to claim 1; and
converting said complex medium.

16. A method for decontamination of a complex medium, comprising:
generating a cold, homogeneous plasma under atmospheric pressure conditions using the device according to claim 1; and
decontaminating at least one pollutant from said complex medium.

17. A method for removal of a material from the surface, comprising:
generating a cold, homogeneous plasma under atmospheric pressure conditions using the device according to claim 1; and
removing said material from said surface by plasma etching.
